(19) United States
(12) Patent Application Publication
(10) Pub. No.: US 2006/0058858 A1
(43) Pub. Date: Mar. 16, 2006

(54) Ambulation actuated pump for generating a thermal load

(76) Inventor: Mark A. Smith, Ann Arbor, MI (US)

Correspondence Address: HARNESS, DICKEY & PIERCE, P.L.C., P.O. BOX 828, BLOOMFIELD HILLS, MI, 48303, US

(21) Appl. No.: 11/226,738

(22) Filed: Sep. 14, 2005

Related U.S. Application Data
(60) Provisional application No. 60/609,806, filed on Sep. 14, 2004.

(51) Int. Cl. A61F 7/00

(52) U.S. Cl. 607/104

(57) ABSTRACT

An apparatus for manipulating the thermal regulatory status of a mammal. The apparatus includes a sealing enclosure for enclosing a portion of the mammal, a heat exchanger operable to transfer energy with the portion of the mammal, and a pumping device operably coupled with at least one of the sealing enclosure and the heat exchanger. The pumping device is actuated through ambulation or movement of the mammal.

### CROSS-REFERENCE TO RELATED APPLICATIONS

This application claims the benefit of U.S. Provisional Application No. 60/609,806, filed on Sep. 14, 2004. The disclosure of the above application is incorporated herein by reference.

### FIELD OF THE INVENTION

The present invention relates to the field of thermal therapeutic applications and, more particularly, to an ambulation actuated pump for generating pressure and/or vacuum for thermal loading.

### BACKGROUND OF THE INVENTION

Human body temperature is normally tightly controlled by an autonomic regulatory system referred to herein as the thermoregulatory system. The most important effector of this regulatory system is blood flow to specialized skin areas where heat from the deep body core can be dissipated to the environment. Normally, when body and/or environmental temperatures are high, the dilation of certain blood vessels favors high blood flow to these surfaces, and as environmental and/or body temperatures fall, vasoconstriction reduces blood flow to these surfaces and minimizes heat loss to the environment.

Elevated deep body core temperature is a problem for many, including athletes, industrial workers, miners, firefighters, and soldiers, and is often associated with exertion. As the temperature of the body's core organs—heart, lungs, liver, kidneys, and brain—rises, fatigue may set in. This fatigue tends to rapidly diminish an individual's strength, endurance, and cognitive functions.

Mammalian thermoregulation principles teach that all mammals have “radiators”—that is, specific regions of the body surface designed for dissipating excess heat from the deep body core to the environment, wherein examples of such include dogs' tongues, rabbits' ears, and the like. In humans, some of these radiator surfaces are found in the palms of the hands and soles of the feet. When an individual gets hot, blood flow naturally increases through these skin regions to dissipate the heat through specialized blood vessels called arteriovenous anastomoses (AVAs).

Conventional methods of cooling the body include remedies typically applied to the skin's surface (i.e., misting fans, ice packs, cold water, etc.). While such solutions often make an individual “feel” cooler temporarily, they are generally ineffective at cooling the body's core organs. This is a result of two phenomena: first, these treatments are applied to the skin's surface, and thus have difficulty penetrating the body's insulating layers of tissue. Second, the cold temperatures of these remedies can result in a vasoconstriction of the peripheral blood vessels and actually cause a reverse of the desired effect by shutting down the natural heat dissipation mechanisms.

Accordingly, there exists a need in the relevant art to provide an apparatus for transferred heat into or out of the thoracic/abdominal core body without triggering concomitant opposing reaction by the thermoregulatory system. Furthermore, there exists a need in the relevant art to provide an apparatus capable of transferring heat into or out of the thoracic/abdominal core body in response to a pumping action created through ambulation. Still further, there exists a need in the relevant art to overcome the disadvantages of the prior art.

### SUMMARY OF THE INVENTION

According to the principles of the present invention, an apparatus for manipulating the thermal regulatory status of a mammal is provided having an advantageous construction. The apparatus includes a sealing enclosure for enclosing a portion of the mammal, a heat exchanger operable to transfer energy with the portion of the mammal, and a pumping device operably coupled with at least one of the sealing enclosure and the heat exchanger. The pumping device is actuated through ambulation or movement of the mammal.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a cross-sectional view illustrating a boot incorporating the ambulation actuated pump of the present invention.

### DETAILED DESCRIPTION OF THE PREFERRED EMBODIMENT

The following description of the preferred embodiment is merely exemplary in nature and is in no way intended to limit the invention, its application, or uses.

The present invention provides an apparatus 10 for dissipating heat from a radiator surface of a mammal in response to a pumping action created through ambulation or varying pressure application to the extremity. Apparatus 10 enhances heat extraction through these radiator surfaces by amplifying local blood flow using carefully controlled temperature settings and/or temperature control. These temperature settings are generally in a range from about 10 degrees Celsius to about 40 degrees Celsius and, more particularly, in a range from about 10 degrees Celsius to about 40 degrees Celsius. The pressure control can provide a negative pressure or an alternating positive and negative pressure. These pressures can generally be in a range of about 5 in. of water to about 35 in. of water.

For individuals or mammalians of any type that are exercising, working, or moving about in extreme environments or those susceptible to heat stress, the present invention helps generally maintain the core body temperature within the zone for optimal performance. When overheated, the present invention serves to cool the body rapidly and non-invasively to reduce fatigue, increase endurance and strength, and improve cognitive function.

By way of background, it should be understood that a significant amount of energy is generated through ambulating, in particular human ambulation. In such cases, when a shoe, boot, or other foot device strikes a surface while walking or moving, energy is consumed through compression and expansion of the sole of the foot device. Until now, this energy is typically lost.

However, according to the present invention, it has been found that by creating or defining a void in a predetermined area(s) of the sole of the foot device, energy that would otherwise be lost during compression and expansion of the sole may be harnessed to move fluids or gas to create pressure differentials within portions of the foot device. That is, by controlling the flow of gas or fluid, a pressure differential may be created inside the shoe or boot. This pressure differential may be used to enhance blood flow to certain vasculature found in the human foot.

A pressure differential created by the pump may also be used to create a temperature differential for the purpose of delivering a thermal load to the foot. Using a pump built into the sole of the shoe or boot or an insert placed in a shoe or boot and the energy generated through ambulation, gas or liquid moved by the pump may be managed for the purpose of expansion and contraction creating relative thermal change in that gas or liquid. The resulting temperature differential can then be used to deliver a thermal load to the foot via a heat exchanger located in close proximity (or in direct contact) with the foot.

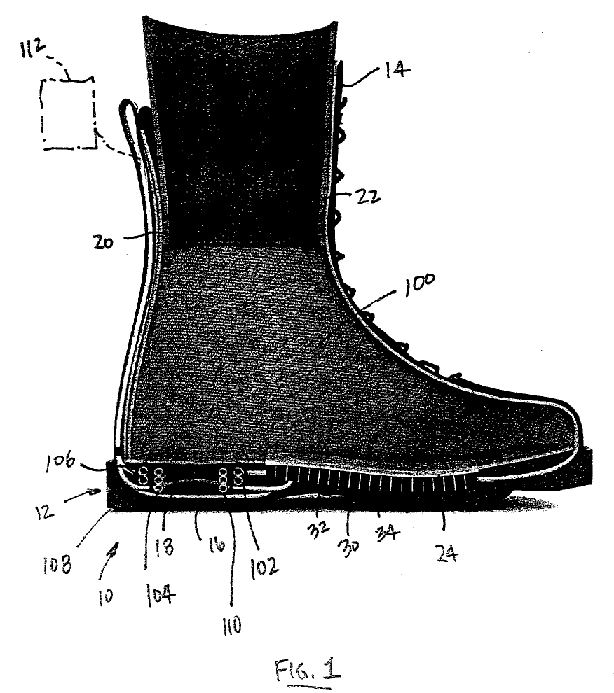

Therefore, according to the principles of the present invention, a device is provided having an advantageous construction. As best seen in FIG. 1, a device 10 is illustrated having a pump 12 coupled within a shoe, boot, or foot device 14. It should be recognized that although foot device 14 of the present disclosure is a boot, any such foot device may be used, such as a shoe, sandal, boot, and the like. Additionally, application of the present invention is not limited to humans, but may be used in connection with any ambulatory mammal. Foot device 14 defines an interior chamber 100, which is adapted to receive a negative pressure or vacuum, a positive pressure, or an alternating positive and negative pressure therein.

Pump 12 is a dual chamber pump disposed at the rear of foot device 14 and is provided for generating differential pressures. Pump 12 includes a low vacuum portion 102 and a high vacuum portion 104 and or a low pressure portion and a high pressure portion.

Low vacuum portion 102 includes a fluid conduit 106 disposed near a heal 108 of foot device 14 that is compressible under load during ambulation. A check valve (not shown) is operably coupled with fluid conduit 106. In one case, the check valve may be operable to create a pressure during positive compression of heal 108. Alternatively, the check valve may be operable to create a vacuum following the positive compression of heal 108—that is, during the relaxing stage of heal 108 following a heal impact. Fluid conduit 106 of low vacuum portion 102 is further coupled to interior chamber 100 of foot device 14. In this regard, low vacuum portion 102 can create a vacuum within interior chamber 100. This vacuum is used to draw blood to the foot for improved cooling as is taught in the following U.S. Pat. Nos. 5,683,438, 6,602,277, 6,656,208, and 6,673,099, which are incorporated herein by reference.

High vacuum portion 104 similarly includes a fluid conduit 110 disposed near heal 108 of foot device 14 that is compressive under load during ambulation. A check valve (not shown) is operably coupled with fluid conduit 110. In one case, the check valve may be operable to create a pressure during positive compression of heal 108. Alternatively, the check valve may be operable to create a vacuum following the positive compression of heal 108—that is, during the relaxing stage of heal 108 following a heal impact. Device 10 may thus be used to generate hot or cold based on the required need of the user. Fluid conduit 110 of high vacuum portion 104 is further coupled to a multi-chamber insert 30.

Mechanical operation of pump 12 is actuated by the ambulation of the leg and foot of the individual and the force generated by the foot striking the ground. As a sole 16 of foot device 14 is compressed under the weight and force of striking a surface, the force generated drives pump 12.

As can be seen in the figure, multi-chamber insert 30 is provided in a position slightly forward from pump 12 and generally under the arch of the foot, which is known as a “radiator” region. Multi-chamber insert 30 includes a plurality of voids 32 and sinters 34. Voids 32 are in fluid communication with high vacuum portion 104 and, thus, are under an extreme pressure differential (positive or negative) for the purpose of generating temperature differential. Sinters 34 are open to the interior chamber 100 of foot device 14.

A thermally conductive material 24 is provided generally above multi-chamber insert 30 to enhance delivery of the thermal load to the bottom of the foot.

In a “dead loss” evaporation system, tubes carry a liquid coolant from a reservoir to the evaporation/expansion chamber of the boot. As the interior temperature of the boot increases a temperature sensitive bimetallic reed valve will open to admit a small amount of liquid. The liquid will be evaporated off to create a temperature change. The temperature change will cause the reed valve to close stopping the admission of liquid.

A bi-metallic reed-valve (not shown) admits an amount of liquid proportional to temperature into a void and or a porous membrane within foot device 14 or boot. A high vacuum is pulled within void 18 causing evaporation of the liquid. This evaporation creates a temperature differential between a heat sink 24 (hot and/or cold conducting surface), which has at least intermittent contact directly or indirectly to the bottom of the foot.

In a closed loop system, liquid or gas will be circulated through a traditional compression and expansion system consisting of an expansion chamber, condenser, heat exchanger and compressor.

Foot device 14 will be fitted with a gas tight seal 20 that may either contact the skin of the user or contact a “mating” material 22 located on a liner or sock so as to cause a gas tight seal at their contact point. This gas tight seal helps maintain a pressure differential between the interior of foot device 14 and ambient.

Furthermore, it is further anticipated that an outer shell 40 of foot device 14 may be used as a “radiator” to dissipate heat. Thus, device 10 may be sealed and operated as a no or limited fluid loss system. Construction of this “radiator” is intended to be incorporated into the actual body of foot device 14. Thermally conductive, permeable, and/or impermeable tubing 46 is used to circulate liquid to indirectly contact ambient conditions. Heat loss or gain may be by convection or conduction.

Furthermore, by allowing evacuation to ambient, device 10 may be built as a “dead loss” system. However, this would require a reservoir of appropriate liquid, generally indicated at 112 in phantom, to be housed within foot device 14 or carried on or attached to the body. Perspiration may act as a supplemental liquid coolant within interior chamber 100 of foot device 14.

It should be understood that additional features, such as solar cells for electrical generation and or micro turbines driven by gas or liquid pumped by the action of ambulation causing actuation of the embedded pump, may further be used. A complimentary device, such as a sock or glove liner that contains thermally conductive material located on the palm of the hand and or the bottom of the foot, may further be used. The sock will be constructed in such a way as to cause a gas tight seal when contacting the accompanying boot or glove.

Furthermore, bifurcation of the vacuum and or pressure device will allow multiple areas of differing pressure and or vacuum within foot device 14.

A similar device may be used for the hand. Using the action of ambulation and the forces generated by the foot and foot device 14. The pressure and vacuum pump 12 housed in foot device 14 could be connected via gas impermeable hoses to a glove that has similar heat sink material located in the palm. The glove or mitten may be housed in a rigid or flexible shell that will not collapse at vacuum's as high as 40 in. of water.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. An apparatus for manipulating the thermal regulatory status of a mammal, said apparatus comprising:
a sealing enclosure for enclosing a portion of said mammal;
a heat exchanger operable to transfer energy with said portion of said mammal; and
a pumping device operably coupled with at least one of said sealing enclosure and said heat exchanger, said pumping device being actuated through ambulation or movement of said mammal.

2. The apparatus according to claim 1 wherein said pumping device comprises:
a low pressure portion having a first fluid conduit, said first fluid conduit being compressible in response to said ambulation or movement of said mammal; and
a high pressure portion having a second fluid conduit, said second fluid conduit being compressible in response to said ambulation or movement of said mammal.

3. The apparatus according to claim 2, further comprising:
a check valve fluidly coupled to said first fluid conduit, said check valve being operable to maintain a desired pressure within said first fluid conduit.

4. The apparatus according to claim 2, further comprising:
a check valve fluidly coupled to said second fluid conduit, said check valve being operable to maintain a desired pressure within said second fluid conduit.

5. The apparatus according to claim 2 wherein said low pressure portion is fluidly coupled with said sealing enclosure to create a vacuum therein.

6. The apparatus according to claim 2 wherein said high pressure portion is fluidly coupled with said heat exchanger.

7. The apparatus according to claim 1 wherein said sealing enclosure comprises:
a housing for enclosing said portion of said mammal;
a fluid tight sealing member being coupled to one of said housing and said portion of said mammal; and
a mating member selectively connecting said fluid tight sealing member to define a fluid tight seal, said mating member being coupled to the other of said housing and said portion of said mammal.

8. The apparatus according to claim 7 wherein said housing is thermally coupled to said heat exchanger and is operable to radiate heat.

9. The apparatus according to claim 1 wherein said pumping device is operable to create a negative, positive, or alternating negative and positive pressure within said sealing enclosure in response to said ambulation of said mammal.

10. The apparatus according to claim 1 wherein said pumping device is operable to create a negative, positive, or alternating negative and positive pressure within said heat exchanger in response to said ambulation of said mammal.

11. The apparatus according to claim 1 wherein said sealing enclosure comprises:
a foot device having a sole, said pumping device being disposed in said sole for actuation in response to compression and or expansion of said sole during ambulation of said mammal.

12. The apparatus according to claim 1 wherein said pumping device is a dual chamber pump.

13. The apparatus according to claim 1 wherein said pumping device is pressurized during a compression stroke of said ambulation or movement of said mammal.

14. The apparatus according to claim 1, further comprising:
a thermally conductive material disposed in thermal contact with said heat exchanger and connectable with at least a portion of said mammal.

15. A method of manipulating the thermal regulatory status of a mammal, said method comprising:
enclosing a portion of said mammal in a sealing enclosure;
operating a pumping device using energy outputted during ambulation of said mammal, said pumping device outputting a positive, negative, or alternating positive and negative pressure; and
operating a heat exchanger in communication with said sealing enclosure in response to said positive, negative, or alternating positive and negative pressure of said pumping device.

16. The method according to claim 15, further comprising:
employing said positive or negative pressure to create a pressure differential within said sealing enclosure relative to ambient.

17. The method according to claim 15 wherein said enclosing a portion of said mammal in a sealing enclosure comprises enclosing a foot of said mammal within a sealable foot device.

18. The method according to claim 15 wherein said operating a pumping device using energy outputted during ambulation of said mammal comprises:
disposing said pumping device within a sole portion of a foot device; and
compressing said pumping device during compression of said sole portion.

19. An apparatus for manipulating the thermal regulatory status of a mammal, said apparatus comprising:
a sealing enclosure for enclosing a portion of said mammal, said sealing enclosure having a sole;
a heat exchanger operable to transfer energy with said portion of said mammal, said heat exchanger being at least partially disposed in said sealing enclosure; and
a pumping device operably coupled with at least one of said sealing enclosure and said heat exchanger, said pumping device being disposed in said sole and actuated at least in part through compression of said sole during ambulation or movement of said mammal.

20. The apparatus according to claim 19 wherein said pumping device comprises:
a low pressure portion having a first fluid conduit, said first fluid conduit being compressible in response to said compression of said sole; and
a high pressure portion having a second fluid conduit, said second fluid conduit being compressible in response to said compression of said sole.

21. The apparatus according to claim 20, further comprising:
a check valve fluidly coupled to said first fluid conduit, said check valve being operable to maintain a desired pressure within said first fluid conduit.

22. The apparatus according to claim 20, further comprising:
a check valve fluidly coupled to said second fluid conduit, said check valve being operable to maintain a desired pressure within said second fluid conduit.

23. The apparatus according to claim 20 wherein said low pressure portion is in fluid communication with said sealing enclosure to create a vacuum therein.

24. The apparatus according to claim 20 wherein said high pressure portion is fluidly coupled with said heat exchanger.

25. The apparatus according to claim 19 wherein said sealing enclosure comprises:
a housing for enclosing said portion of said mammal;
a fluid tight sealing member being coupled to one of said housing and said portion of said mammal; and
a mating member selectively connecting said fluid tight sealing member to define a fluid tight seal, said mating member being coupled to the other of said housing and said portion of said mammal.

26. The apparatus according to claim 25 wherein said housing is thermally coupled to said heat exchanger and is operable to radiate heat.

27. The apparatus according to claim 19 wherein said pumping device is operable to create a negative, positive, or alternating negative and positive pressure within said sealing enclosure in response to said compression of said sole.

28. The apparatus according to claim 19 wherein said pumping device is operable to create a negative, positive, or alternating negative and positive pressure within said heat exchanger in response to said compression of said sole.

29. The apparatus according to claim 19 wherein said pumping device is pressurized during a compression stroke of said compression of said sole during ambulation or movement of said mammal.

30. The apparatus according to claim 19 wherein said pumping device is pressurized during a relaxing stroke of said compression of said sole during ambulation or movement of said mammal.
